(19) United States
(12) Patent Application Publication
(10) Pub. No.: US 2025/0325520 A1
(43) Pub. Date: Oct. 23, 2025

(54) Methods for Targeting Cancer Cells Using Amino Acids

(71) Applicant: University of Denver, Denver, CO (US)

(72) Inventor: Dali Sun, Denver, CO (US)

(73) Assignee: University of Denver, Denver, CO (US)

(21) Appl. No.: 19/179,845

(22) Filed: Apr. 15, 2025

Related U.S. Application Data
(60) Provisional application No. 63/635,619, filed on Apr. 18, 2024.

(51) Int. Cl. A61K 31/4172; A61K 31/198; A61K 31/7068; A61K 45/06; A61P 35/00; C12N 9/99; G01N 33/50; G01N 33/68

(52) U.S. Cl. CPC A61K 31/4172; A61K 31/198; A61K 31/7068; A61K 45/06; A61P 35/00; C12N 9/99; G01N 33/5044; G01N 33/6842; G01N 33/6893; G01N 2800/52

(57) ABSTRACT

A method for selectively targeting tumor cells for a given strain of cancer includes providing amino acids shown to discourage promotion of tumor growth for the given strain of cancer. The method may include determining the amino acids by identifying extracellular vesicles (EVs) involved in cell-cell signaling for tumor development, quantifying relative abundance of amino acids present in patient samples, comparing the relative abundance of the amino acids from the patient sample to a known distribution of the individual amino acids present in a nonmalignant sample, and comparing the abundance of the amino acids in the patient sample to the nonmalignant sample to determine the plurality of amino acids suitable for treating the given strain of cancer. The method may include supplementing the combination of the plurality of amino acids with a chemotherapy drug.

### REFERENCE TO RELATED APPLICATIONS

The present application claims the benefit of U.S. Provisional Pat. App. No. 63/635,619, filed 2024 Apr. 18 and titled “Methods for Targeting Cancer Cells Using Amino Acids,” which referenced application is incorporated hereby in its entirety by reference.

### SPONSORED RESEARCH AND DEVELOPMENT

This invention was made with government support under contracts R03CA252783, R21CA270748, and U54GM128729 awarded by the National Institutes of Health. The government has certain rights in the invention.

### FIELD OF THE INVENTION

The present invention relates to cancer treatment. In particular, but not by way of limitation, the present invention relates to methods for cancer treatment by identifying and targeting specific molecules.

### DESCRIPTION OF RELATED ART

Currently available cancer therapies generally affect both cancer cells and normal tissue cells, thus leading to severe side effects and patient suffering while offering limited efficacy.

Thus, there is a need for innovative and selective cancer treatment methods to specifically target the tumor cells to improve prognosis and limit side effects for the patient.

### SUMMARY OF THE INVENTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

In an embodiment, a method for treating cancer includes providing one or more selected amino acids to elicit selective toxicity to tumor cells.

In a further embodiment, a method for selectively killing targeted tumorous cells in a patient includes: 1) identifying one or more amino acids in proteins preferentially excreted by extracellular vesicles associated with the targeted tumorous cells; and 2) supplementing the patient with the one or more amino acids so identified.

In embodiments, the targeted tumorous cells are pancreatic ductile adenocarcinoma (PDAC), and the one or more amino acids includes at least one of isoleucine and histidine.

In embodiments, the method wherein the supplementing is performed as an adjuvant treatment along with providing a standard chemotherapy, such as gemcitabine treatment.

In an embodiment, a method for selectively targeting tumor cells for a given strain of cancer includes providing a combination of a plurality of amino acids shown to discourage promotion of tumor growth for the given strain of cancer.

In certain embodiments, the method further includes: determining the plurality of amino acids by identifying extracellular vesicles (EVs) involved in cell-cell signaling for tumor development for the given strain of cancer, performing a proteomics analysis of the EVs in a patient sample to quantify relative abundance of individual amino acids present in the patient sample, comparing the relative abundance of the individual amino acids from the patient sample to a known distribution of the individual amino acids present in a nonmalignant sample, and if a significant imbalance in the relative abundance of the individual amino acids is present in the patient sample in comparison to the nonmalignant sample, then determining the individual amino acids exhibiting the significant imbalance as the plurality of amino acids suitable for discouraging promotion of tumor growth for the given strain of cancer.

In embodiments, the method further includes supplementing the combination of the plurality of amino acids with a chemotherapy drug.

In certain embodiments, the given strain of cancer is pancreatic ductile adenocarcinoma (PDAC). In embodiments, the combination of the plurality of amino acids targets elimination of exoribonuclease XRN1. In embodiments, the plurality of amino acids includes at least histidine and isoleucine. In certain embodiments, the method further includes supplementing the combination of the plurality of amino acids with gemcitabine.

In certain embodiments, a therapeutic index (TI) of each one (i) of the plurality of amino acids is determined by testing a plurality of cell lines for the given strain of cancer, and calculating the TI using an equation:

$${TI}_{i} = \frac{{\sum }_{1}^{n} ( 1 - {v}_{i}^{Tumor} )}{n} - ( 1 - {v}_{i}^{N o n - t u m o r} ) ,$$

where

$${v}_{i}^{Non - tumor}$$

is maximum cell viability value when treating nontumor tissue cells,

$${v}_{i}^{T u m o r}$$

is maximum cell viability value when treating tumor cells, and n is a total number of cell lines tested for the given strain of cancer.

In embodiments, a method for identifying presence of a given strain of cancer includes identifying extracellular vesicles (EVs) involved in cell-cell signaling for tumor development for the given strain of cancer, performing a proteomics analysis of the EVs in a patient sample to quantify relative abundance of individual amino acids present in the patient sample, comparing the relative abundance of the individual amino acids from the patient sample to a known distribution of the individual amino acids present in a nonmalignant sample, and if a significant imbalance in the relative abundance of the individual amino acids is present in the patient sample in comparison to the nonmalignant sample, then determining the presence of the given strain of cancer.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a,’ ‘an,’ and ‘the’ include plural referents unless the context clearly dictates otherwise.

### BRIEF DESCRIPTION OF DRAWINGS

FIG. 1 shows a protein Venn diagram and amino acid distribution relevant to the methods described herein.

FIG. 2 shows a graph illustrating the percentage difference of amino acids between cell and extracellular vesicle (EV), as relevant to the methods described herein.

FIG. 3 shows a graph ranking of different amino acids by exocytosis selectivity in tumor cells, as relevant to the methods described herein.

FIG. 4 shows a simplified illustration of an amino acid treatment method of tumor cells, in accordance with embodiments.

FIG. 5 shows a graph illustrating a comparison of the area under curve (AUC) and therapeutic index (TI) obtained using amino acids cytotoxicity tests, in accordance with embodiments.

FIG. 6 shows a bar graph illustrating the pH change caused by the amino acid treatment method described herein, in accordance with embodiments.

FIGS. 7 and 8 show graphs illustrating the viability dose response of two different patients (individuals H and I) within the same cell lineage, in accordance with embodiments.

FIGS. 9 and 10 show graphs comparing the cell viability dose-response of Land D to form amino acids.

FIG. 11 shows a color chart illustrating the synergetic efficacy of combining histidine (H) and isoleucine (I) treatments, in accordance with embodiments.

FIG. 12 shows graphs comparing the cytotoxicity of mixing a variety of treatments, in accordance with embodiments.

FIG. 13 shows bar graphs comparing the treatment efficacy of AA (histidine and isoleucine) and GEM (gemcitabine) treatments, in accordance with embodiments.

FIG. 14 shows a graph illustrating the adjuvant efficacy of combined AA and GEM treatments on tumor cells, in accordance with embodiments.

FIG. 15 shows a bar graph comparing the flow cytometric analyses for different treatment conditions, in accordance with embodiments.

FIGS. 16-19 show the measured counts corresponding to the AUC characterizations for NT, H, I, and AA, respectively, as summarized in FIG. 15.

FIG. 20 shows a bar graph comparing the flow cytometric analyses for different treatment conditions, in accordance with certain embodiments.

FIGS. 21-24 show the measured counts corresponding to the AUC characterizations for NT, H, I, and AA, respectively, as summarized in FIG. 20.

FIG. 25 illustrates cell morphology changes on tumor and nontumor cells as a result of AA treatments, in accordance with embodiments.

FIG. 26 shows graphs comparing the mitochondrial activity in treated and non-treated tumor cells, in accordance with embodiments.

FIG. 27 shows a bar graph comparing the difference in enrichment analysis before and after AA treatment, in accordance with embodiments.

FIG. 28 shows a graphical summary of the results of reactome analyses before and after AA treatment, in accordance with embodiments.

FIG. 29 shows bar graphs comparing the Western blot expression analysis results for tumor and nontumor cells with combined AA treatment, in accordance with embodiments.

FIG. 30 shows graphs comparing the cytotoxicity effects of AA treatment XRN1 knockdown and wild type cells, in accordance with embodiments.

FIG. 31 shows a simplified schematic of the in vivo experimental design for validation of the treatment method, in accordance with embodiments.

FIGS. 32 and 33 show graphs comparing tumor volume changes among different treatment conditions, in accordance with embodiments.

FIG. 34 shows a graph comparing the survival rate among various treatment conditions, in accordance with embodiments.

FIG. 35 shows a bar graph comparing the percentage necrosis derived from histology image analysis after different treatments, in accordance with embodiments.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION OF THE INVENTION

As noted above, current cancer therapies have limited efficacy and significant side effects. For instance, it is essentially impossible to target only the cancerous cells using conventional methods such as chemotherapy and radiation therapy, which leads to the nonspecific destruction of both cancerous and non-cancerous cells in the patient. Thus, existing cancer treatment methods often cause severe side effects that lead to patient suffering. Therefore, innovative and selective treatment methods of cancer are urgently needed.

Certain cancers are particularly problematic to treat. For example, pancreatic ductile adenocarcinoma (PDAC) is one of the deadliest types of cancer, characterized by extremely limited therapeutic options and a poor prognosis, as it is often diagnosed during later stages of the disease. Pancreatic ductal adenocarcinoma (PDAC) is the 4th leading cause of cancer death in the United States and the eighth leading cause worldwide, with an increasing incidence [1-3]. Treatment options for pancreatic cancer are limited to surgery, chemotherapy, radiation therapy, and palliative care [4]. Although surgical resection can potentially ameliorate early-stage disease, more than 80% of patients present with locally advanced or metastatic disease at the time of diagnosis are ineligible for resection[4]. While chemoradiation and systemic chemotherapy are the mainstays of treatment to slow disease progression for PDAC disease at advanced stages [5], these therapies attack all rapidly dividing cells, including healthy cells, and thus cause significant side effects and toxicity [6]. Thus, the lack of selective protection for healthy cells has limited the effectiveness of current therapies, particularly against pancreatic cancer. These challenges underscore the need for innovative and more effective treatments to achieve breakthroughs in pancreatic cancer treatment.

Recent research has identified the crucial role of extracellular vesicles (EVs) in cell-cell signaling and selective transfer of cellular information for tumor development [7-11]. These membrane vesicles of endocytic origin, with sizes ranging from 30 to 150 nm, contain biomolecules, such as DNA, RNA, and proteins [12], which are capable of migrating between cells. EVs have been found to play a critical role in promoting the cascade of cell signaling events that lead to tumor growth, invasion, and metastasis. Studies have shown that the cargoes carried by EVs, especially proteins, and DNA (e.g., secondary protein structures, excessive mitochondrial DNA) have an uneven distribution in tumor-derived EVs [13-17].

Significantly, through proteomics analyses of EVs, it is recognized herein that PDAC tumor cells secrete an imbalanced distribution of amino acids. In particular, results of proteomics analysis of EVs to quantify the abundance of individual amino acids in PDAC patients indicate that the proteins may then be linked with their primary sequences [19] for statistical analysis of the relative abundance of the individual amino acids that make the protein. PDAC cells appear to preferentially excrete proteins with certain amino acids via the EVs, including isoleucine (I) and histidine (H). These preferential amino acids are likely associated with disease progression and may be targeted to elicit selective toxicity to PDAC tumor cells while sparing normal cells. By comparing the individual amino acid abundance between proteins of PDAC tumors and nontumor cells at both EV and cellular levels, it is recognized herein that there exists a significant imbalance in the amino acids distribution between PDAC tumor cells and EVs, although not in nonmalignant counterparts.

Further, it is recognized herein that EVs have significant potential as novel biomarkers for noninvasive cancer detection. Thus, EVs are a promising information source for developing markedly improved therapies for PDAC and other types of cancers. Whereas the current limitations associated with therapies for PDAC have been linked to the high aggressiveness of the disease and related cascade of cell signaling events that promote tumor growth, invasion, and metastasis in PDAC [18], EV-based approaches provide a path toward more targeted and effective treatment of aggressive cancers such as PDAC.

In vitro and in vivo experiments have been performed to demonstrate that supplementation with specific amino acids effectively eliminates PDAC cells. Further, the exoribonuclease XRN1 has been mechanistically determined as a potential target for these amino acids.

The high selectivity of the presently described treatment method enables specific targeting of tumor metabolism with very low toxicity to normal, nontumor cells. Moreover, this treatment approach is easy to administer and provides sustained tumor-killing effects. That is, it is recognized herein that exocytosed amino acids may serve as therapeutic targets for designing treatments of a variety of types of cancers, including intractable PDAC.

The above recognition and results suggest that the tumor cells selectively exocytose certain amino acids through EVs. This discovery regarding EVs led to the recognition that cancerous tumors may be stressed with amino acids of high exocytosis selectivity. Further, combined treatment of amino acids identified from selective exocytosis of PDAC cells via EVs, mediates necrosis of PDAC cells in vitro and in vivo. Moreover, the potential therapeutic target of the combined amino acids treatment has been identified to be the XRN1.

XRN1 is a 3′-5′ exoribonuclease that is involved in the cytoplasmic mRNA decay and stability in cellular processes critical to some disease development, such as osteosarcoma and Wilms' tumor [20]. Previously, it was reported that XRN1 silencing in melanoma cells suppressed RNA decay and stimulated antitumor immunity [21]. It is further recognized herein that targeting the XRN1 gene with amino acids may be an effective approach for the selective treatment of PDAC tumors by affecting the RNA degradation pathway.

The present disclosure describes an amino acid-based treatment of cancerous tumors, such as PDAC tumors. This approach has been developed using innovative proteomics analysis that accurately quantifies individual amino acids in EVs derived from PDAC cells compared to those from normal cells. The combined amino acid treatment described herein enables a non-starvation treatment approach that is selective for killing PDAC tumors with minimal side effects on nontumor cells. The recognitions and approaches described herein may establish a new paradigm for using EVs information in drug discovery, contribute to an improved understanding of the molecular mechanism underlying PDAC development, provide the basis for developing selective and effective therapeutic interventions against PDAC, and offer translational advantages for clinical applications.

### Example Embodiment

Malignant pancreatic cell lines (PANC-1 and MIA PaCa-2) and nonmalignant human pancreas cell line (HPNE) were obtained from the American Type Culture Collection (Manassas, Virginia). The cells were cultured in DMEM medium (HYCLONE™ sera, available from GE Healthcare Life Sciences) with HPNE cells having 0.1 ng/ml EGF (Novus Biologicals, USA) included. All cultures were supplemented with 10% fetal bovine serum (FBS, Life Technology, Thermo Fisher Scientific Inc.), penicillin (1 U), and streptomycin (1 μg/ml). All cells were maintained in a humidified incubator with 5% CO₂ at 37° C. All cell lines were cultured in triplicate under the same conditions, then harvested to collect independent exosome samples.

Cells were seeded in 96-well plates at a density of 104 cells/well. After 24 hours, culture medium with increasing concentrations of treatment was added to the treatment group for 48 hours. Cell viability was accessed by Cell Counting Kit-8 (CCK-8; Dojindo Laboratories) following the manufacturer's instructions. Briefly, a mixture of 10 μl of CCK-8 and 190 μl media was added into each well and the cells were incubated for another 1 hour. The absorbance of each well was measured at 450 nm using a microplate reader. Each experiment was repeated six times.

Mitochondrial mass quantification was performed by flowcytometry. Cells were stained using MITOVIEW™ Green mitochondrial dye (Biotium, Inc.). Briefly, the cells in the culture dish were treated with 2 ml of Trypsin-EDTA solution for 3 minutes at 37° C., followed by the addition of 2 ml growth medium. The cell suspensions were centrifuged to obtain the cell pellet and washed three times with phosphate buffered saline (PBS) before being incubated for 20 minutes at 37° C. in 200 nM dye in the medium. Cells were then subjected to flow cytometric analysis using a BD ACCURI™ C6 Fflow cytometer (BD Bioscience). At least 10,000 cells were acquired from each sample. A fluorescein isothiocyanate (FITC) channel was used to capture the signal from the green dye. Flowing Software (Turku Centre for Biotechnology) was used for the analysis of the cytometric data. The intensity was normalized to the mean of the control.

The quantitative proteomics studies of the treated and untreated cells were performed. Briefly, four replicates each for cell lysates were diluted to 1 μg/μl with 100 mM NH₄HCO₃ supplemented with 10 mM dithiothreitol, incubated at 37° C. for 1 hour, then mixed with 30 mM iodoacetamide, incubated in the dark for 30 minutes at room temperature before overnight digestion with 1 μg trypsin at 37° C. Digestions were terminated by the addition of 0.1% trifluoroacetic acid and diluted to 0.25 μg/μl protein with H₂O/acetonitrile (95:5), centrifuged at 21,000 g for 20 minutes. The digested peptides were cleaned up using stop-and-go-extraction tips (StageTips). The dried peptides were reconstituted with H₂O/acetonitrile (95:5) before liquid chromatography with tandem mass spectrometry (LC-MS-MS) analyses using approximately 500 ng peptide/injection. Samples were analyzed using Q Exactive™ HF-X Quadrupole-Orbitrap™ mass spectrometer system (Thermo Fisher Scientific). The peptides were eluted with a linear gradient from 2.5 to 35% buffer B (80% acetonitrile (ACN) in 0.1% formic acid (FA)) over 45 minutes. Following the linear separation, the system was ramped up to 75% in the next 10 minutes, followed by 100% in 2 minutes. Then it was re-equilibrated to 2.5% in 7 minutes. The MS1 scans were collected from 300-1650 m/z with an automatic gain control (AGC) target of 3E6, a resolution of 60,000 at 200 m/z, and followed by a top-15 MS2 loop. MS/MS scans were collected with a resolution of 15,000 at 200 m/z, with an AGC target of 1E5 and a maximum injection time of 118 ms. The dynamic exclusion time was set for 30 seconds.

All LC-MS/MS data were analyzed by label-free quantitation mode and searched using the Sequest HT algorithm within PROTEOME DISCOVERER™ 2.4 software (Thermo Fisher Scientific) against Homo sapiens proteome database (UP000005640) to obtain peptide and protein identifications using a precursor mass tolerance of 10 ppm and fragment mass tolerance of 0.02 Da. For all searches, trypsin was specified as the enzyme for protein cleavage, allowing up to two missed cleavages. Oxidation (M) and carbamidomethylation (C) were set as dynamic and fixed modifications, respectively. The peptide spectrum match and protein false discovery rate (FDR) was set to 0.01 and determined using a percolator node. Relative protein quantification of the proteins was performed using the Minora feature detector node with default settings using peptide spectrum matches (PSM). The intensity ratio and adjusted p-values were calculated and provided in the supplementary file 2 (Appendix 2). Also, the proteomics data have been deposited to the ProteomeXchange Consortium via the PRIDE59 partner repository with the dataset identifier PXD047958. The proteomics data used for uncovering the unbalanced distribution of the amino acids was adopted from our previous research [22,60].

Determination of the therapeutic index of each amino acid

The therapeutic index of each amino acid was determined. The therapeutic index (TI) of a given amino acid (i) is defined as:

$${TI}_{i} = \frac{{\sum }_{1}^{n} ( 1 - {v}_{i}^{Tumor} )}{n} - ( 1 - {v}_{i}^{N o n - t u m o r} ) \\ where {v}_{i}^{Non - tumor} and {v}_{i}^{T u m o r} \qquad ( Eq . 1 )$$

are the maximum cell viability when treating the nontumor tissue cells (HPNE) and tumor cells, respectively, n being the number of PDAC cell lines tested [61].

Assays to assess cell viability, apoptosis, and necrosis was performed. 20-30% confluent cells were treated with branch chain amino acids, histidine, and isoleucine, and incubated at recommended humidity (5%) and temperature (37° C.) for 48 hours. Four separate groups of treatments were considered: (1) No treatment (NT); (2) Histidine only (H); (3) Isoleucine only (I); and (4) Both Histidine+Isoleucine (AA). After 48 hours, adherent and non-adherent cells were collected and counted using a hemocytometer to obtain 5×10⁵ cells per tube. We used Abcam Apoptosis/Necrosis Assay kit (Catalog: ab176749, Abcam). Cells were centrifuged at 500 xg for 5 minutes at 4° C. The supernatant was discarded, and the cell pellet was resuspended in 200 μl assay buffer. For detecting viable cells, we added 1 μl CytoCalcein™ Violet 450 fluorogenic dye in each tube. For apoptosis and necrosis, 2 μl of Apopoxin™ Green indicator and 1 μl of 7-Aminoactinomycin D (7-AAD) were added, respectively. Next, cells were incubated for 60 minutes at room temperature. An additional 300 μl assay buffer was added to each tube, and samples were analyzed in a BD FACSMelody™ cell sorter. Results were analyzed in FlowJo™ software (Version: 5.2) for flow cytometry, and changes are expressed as median fluorescent intensity.

Immunoblotting analyses were then performed. Cells with 20-30% confluency were treated with branch chain amino acid for 48 hours at 5% humidity and 37° C. After treatment, the media was discarded, and PBS was used to wash the cells twice before adding 100 μl of 2× cell lysis buffer (Catalog: 9803S, Bio-Rad Laboratories). Cells were scrapped, collected, and mixed in a 360° rotator for 30 minutes at 4° C. Cell lysates were centrifuged at 15000 rpm for 15 minutes at 4° C. The supernatant was collected, and the protein concentration was determined using the bicinchoninic acid (BCA) Protein Assay Kit (Catalog: 23225, Thermo Fisher Scientific). 30 μg of total protein from each cell lysate was mixed with an equal volume of 2× SDS Laemmli sample buffer (Catalog: 1610737, Bio-Rad) containing β-Mercaptoethanol and boiled at 100° C. for 5 min. Then the sample was loaded into 4-20% precast polyacrylamide gel (Catalog: 4561093; Bio-Rad) for electrophoresis. After separating, the protein samples were transferred to the polyvinylidene fluoride (PVDF) membrane (Catalog: 88520; Thermo Fisher Scientific) using the Trans-Blot Turbo transfer system. Then, the membrane was blocked using 5% non-fat dry milk (Catalog: SC-2325; Santa Cruz Biotechnology) in tris-buffered saline with TWEEN® 20 (TBST) for 2 hours. The membrane was washed 3 times in TBST solution and incubated with primary antibody, XRN1 (Dilution: 1:2500, Cat: ab70259, Abcam) overnight at 4° C. The next day, the membrane was washed and treated with an HRP-tagged secondary antibody for 2 hours at room temperature. After washing, the protein was visualized using Pierce™ ECL 2 Western blotting substrate (Catalog: PI80196) in the Western blotting detection system. ImageJ analysis software was used to quantify the expression level of proteins. To dilute both primary and secondary antibodies, 5% bovine serum albumin (BSA; Catalog: BP1600, Fisher Scientific) in TBST was used. The dilution ratio was optimized before performing the experiments.

A shRNA transfection analysis was performed. PANC-1, XRN1 knockdown cells were generated using short hairpin RNA (shRNA) plasmid (catalog: TF300419C, OriGene). 40-50% confluent cells in 6-well plates were transfected using TURBOFECTIN™ 8.0 reagent (Catalog: TF81001, OriGene). A complex solution of TURBOFECTIN™ 8.0 reagent and shRNA plasmid was prepared before transfection. For each well in 6-well plates, 250 μl of Dulbecco's Modified Eagle Medium (DMEM), 2 μg of shRNA plasmid DNA, and 3 μl of TURBOFECTIN™ 8.0 reagent was mixed and incubated at room temperature for 15 minutes. After that complex solution was added to the well and incubated for 24 hours at 37° C. 24 hours post transcription, the cells were moved into the serum-containing medium with 1 μg/ml puromycin for selection. Once all of the non-transfected cells were dead, cells were moved into a cell culture dish and grown in a medium containing 1 μg/ml puromycin. Each individual colony was picked up using cloning cylinders. Immunoblotting was performed to confirm the percent knockdown of XRN1 expression in clones. Glyceraldehyde-3-phosphate dehydrogenase (GAPDH) expression is used as the loading control. Control shRNA provided in the kit was used to transfect control cells.

The treatment efficacy was validated in animal experiments. In particular, the xenograft model was established with six to eight-week-old male nude mice (J:NU-Foxn1nu) purchased from The Jackson Laboratory (Bar Harbor, ME). Five mice were housed per cage in static disposable cages from Innovive (San Diego, CA). The mice's food and water were checked daily, and housing was changed weekly. The pancreatic cancer tumor was injected subcutaneously into the left flank of each mouse with 4×106 PANC-1 cells suspended in 100 μl of serum-free media with 50% MATRIGEL® matrix to establish a subcutaneous pancreatic tumor.

Mice were observed three times daily for the first seven days and tumor xenograft was confirmed by the presence of the tumor. After confirmation, mice were divided into four groups randomly: three treatment groups (1)AA (histidine and isoleucine), (2) AA+GEM (histidine and isoleucine and gemcitabine), and (3) GEM (gemcitabine) and one control group (4) phosphate buffered saline (PBS). Tumor volume and mice weight were measured every fifth day. Tumor volume was established by measuring length and width; the volume formula used was the modified ellipsoid volume formula (½ length×width 2).

For AA and control groups, treatment was given by oral gavage. AA treatment was made from a combination of histidine (44 mg/ml) and isoleucine (22 mg/ml) dissolved in PBS. For the two groups with AA, oral gavage treatment was administered daily with 200 μl AA treatment using a 20 ga polypropylene feeding tube attached to a syringe. For the control group, oral gavage treatment was administered using the same method except with 200 μl PBS. For the GEM and AA+GEM groups, GEM was given by intraperitoneal injection. GEM groups were injected twice a week with 200 μl of GEM (15 mg/ml) dissolved in PBS. All treatments were administered for sixty days. Two days after the last treatment dose, the mice were sacrificed using an isoflurane overdose and cervical dislocation. Tumors were carefully excised using scissors and a scalpel and placed on a blank sheet to be photographed.

Enrichment analysis and pathway identification was performed. The Reactome Pathway Database (https://reactome.org/) entails molecular and pathological details of processes in various diseases. Reactome was used to evaluate the enrichment analysis and determine the pathway affected by the treatment following the documentation procedure on the reference website[62].

Histological analysis was performed in the following manner. Tumor sections were stained with hematoxylin and eosin (H&E) for image analysis. The stained tumor tissue sections on the slides were analyzed using QuPath digital pathology analysis software version 0.4.3 [41]. Briefly, the tumor image analysis was carried out by creating a workflow to detect necrotic cells based on H&E staining reflected on the tissue slide. The slide image was optimized for staining. Annotation and objects that distinguish necrotic tumor cells were created as a consequence of the staining and annotated accordingly. A training classifier was then used to model the workflow before applying the detection to the whole tissue section.

Data analysis comparisons between the two groups were performed using an unpaired two-sample t-test. All data analysis was performed using OriginPro software from OriginLab. Data are presented either as representative examples or means±standard error of the mean (SEM) of 3+experiments. All comparison groups had equivalent variances. A p value<0.05 was viewed to be statistically significant. As used herein, * represents p<0.05, ** represents p<0.01, *** represents p<0.001, and * ** represents p<0.0001.

Selective exocytosis of amino acids in PDAC cell-derived EVs is discovered via quantitative proteomics profiling.

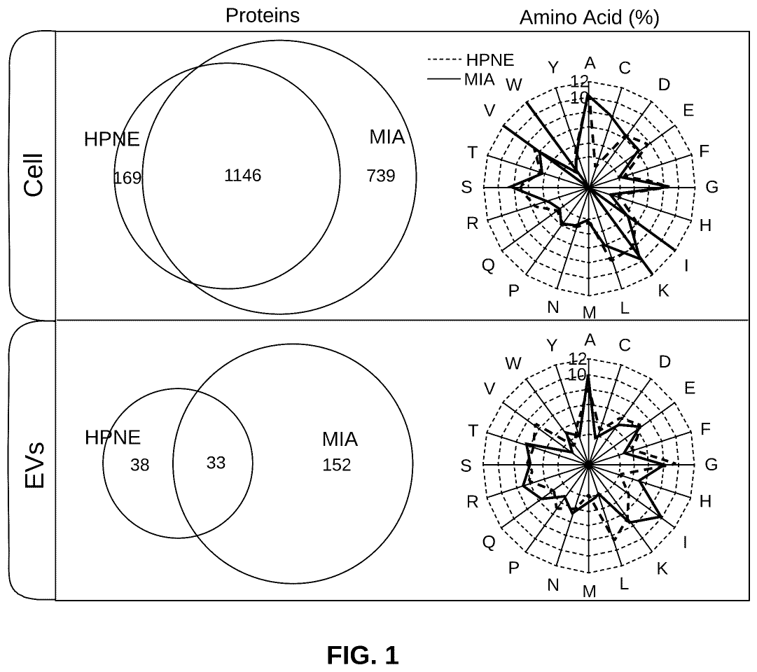

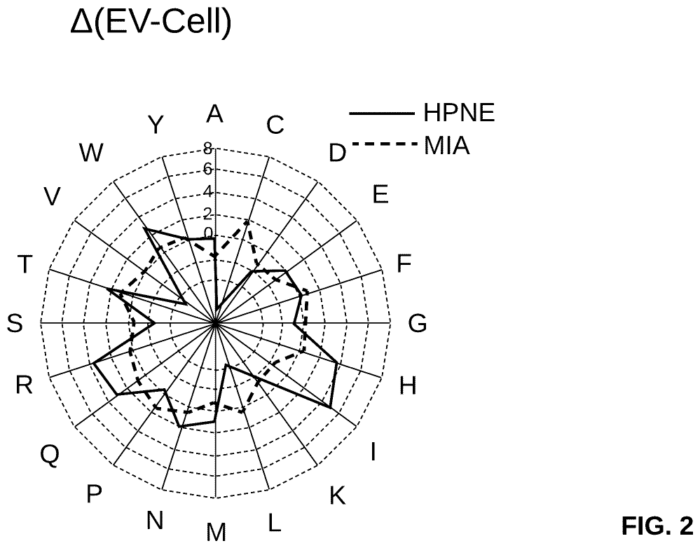

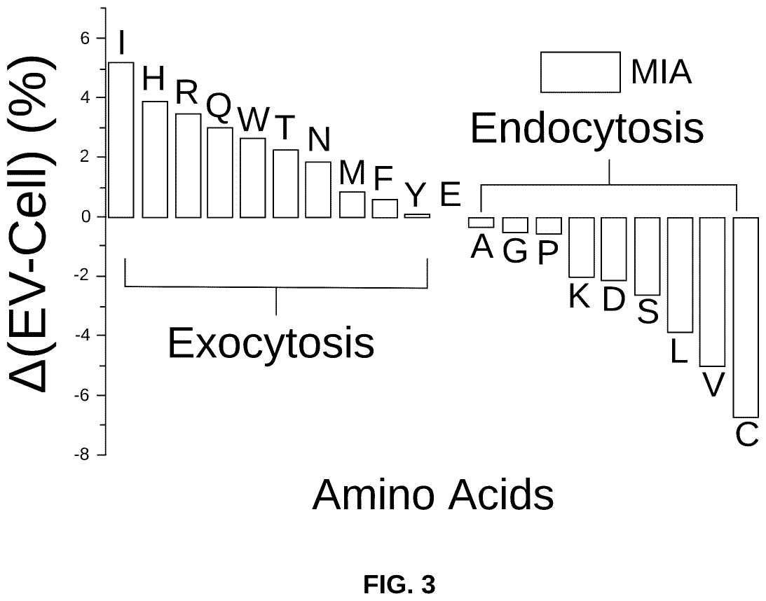

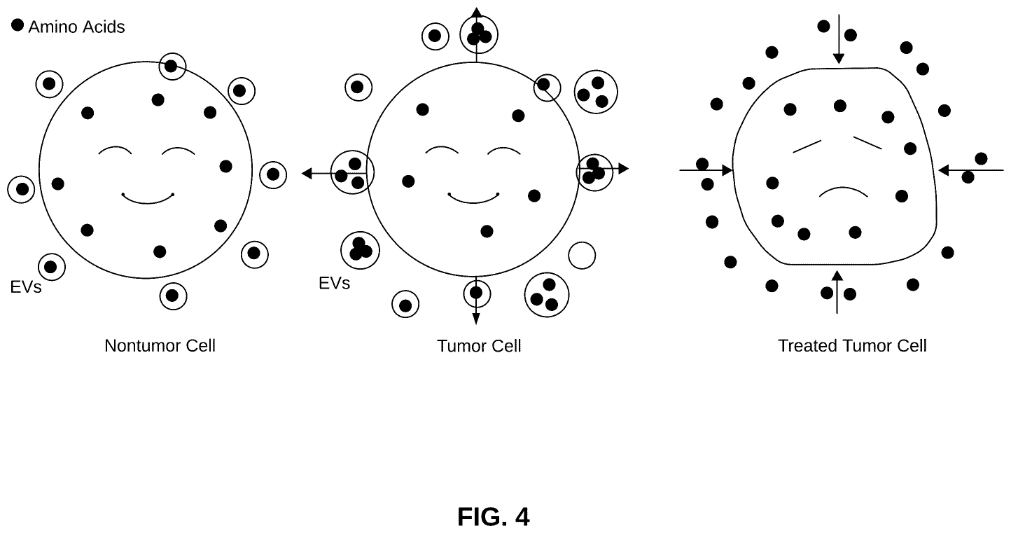

Modern mass spectrometry-based protein profiling provides both individual identification of proteins and quantitative information on each protein in a given sample. This functionality has facilitated comparative proteomics to identify and quantify proteins in cells from tumor and nontumor tissues at both the cellular and EV levels. In particular, FIG. 1 shows results of proteomic analysis of cellular and EVs level amino acid distribution. EVs: Extracellular vesicles, where “HPNE” corresponds to hTert-HPNE (nonmalignant pancreas epithelial) and “MIA” corresponds to MIA PaCa-2. (malignant pancreas epithelial). FIG. 2 shows percentage difference (Δ) of amino acids between cells and EVs. FIG. 3 shows exocytosis selectivity ranking of amino acids (Δ) in tumor cells, using single letter representation of the amino acids. Finally, FIG. 4 shows a schematic representation of amino acids treatment inspired by EVs exocytosis.

Comparative proteomics studies of both cell and EVs of PDAC and immortalized nontumor human ductal epithelial cells, respectively, have been performed [22]. The proteins were evaluated by determining the relative abundances of each identified protein within tumor and nontumor cells and EVs. It was then recognized that the percentage of the proteins shared by tumor and nontumor tissue cells was significantly higher at the cellular level than at the EVs level (FIG. 1), indicating possible selective exocytosis in EVs shedding. Previous studies have indicated that protein transport complexes may be involved in sorting proteins and other

biomolecules in EVs before shedding occurs [23]. However, the subsequent sorted protein

distributions specific to PDAC cells have not been comprehensively evaluated [21]. Therefore, the present study described herein further investigated the differential protein distribution down to the single amino acid level [24].

To further evaluate the proteomics results, a method to study the statistical distribution of the amino acids was developed. The accession identifiers of identified proteins were used to retrieve their primary amino acid sequences from the UNIPROT® database (https://www.uniprot.org/). Thus, a detailed analysis of the amino acid content of each protein identified in the study was performed. The resulting percentage of i-th amino acid in the sample was defined as

$${p}_{i} = {\sum }_{1}^{n} {a}_{i} \cdot {a}_{i} ,$$

where a is the amino acid percentage calculated by the primary sequence in the protein, q is the relative abundance of the protein in the sample, n is the number of the proteins identified. By evaluating the amino acid distribution at the cellular and EVs level, it was recognized that at the cellular level, the distributions in the tumor and nontumor tissue cells are analogous except for cysteine (FIG. 1). However, the distribution varied from tumor to nontumor cells at the EVs level, which implied selective exocytosis of amino acids through tumor EVs. To elucidate the selectivity, the i-th amino acid percentage difference between EVs and cellular level is defined as

$${Δ}_{i} = {p}_{i}^{E V} - {p}_{i}^{c e l l} , where {p}_{i}^{E V} and {p}_{i}^{c e l l}$$

are the amino acid percentage at EVs and cellular levels, respectively. The selectivity (Δ) of amino acids in tumor and nontumor cells is summarized in FIG. 2.

As may be seen in FIG. 2, the nontumor cells featured homogeneous selectivity over all amino acids in a round profile. However tumor cells present imbalanced selectivity, which implicates selective exocytosis of specific amino acids in tumor cells through EVs. The amino acids are ranked by their A value in the tumor cells in FIG. 3, revealing that amino acids with positive Δ values (PSA) may undergo selective exocytosis processes, thus indicating the metabolic disparity of the amino acids in tumor cells. As may be seen in FIG. 3, isoleucine (I), histidine (H), and arginine (R) are the top three amino acids in terms of exocytosis, while cysteine (C), valine (V), and leucine (L) are the top in terms of endocytosis.

The knowledge gained using the study results above may be applied in treating certain types of cancers. Metabolic reprogramming is a hallmark of cancer, and PDAC tumors display distinct metabolic alterations due to the activation of Kirsten rat sarcoma viral oncogene homolog (KRAS) mutation to meet the growth demands in relatively hypoxic and nutrient-poor niches [25,26]. Approximately 90% of PDAC tumors exhibit KRAS mutations, and KRAS-induced metabolic alterations rely on amino acids to provide the carbon and nitrogen sources to meet the energy requirements for PDAC growth and survival [27]. For instance, KRAS-mediated alteration in protein expressions, such as repression of glutamate dehydrogenase (GDH) and upregulation of cytoplasmic aspartate transaminase (GOT1), are known to impact glutamine metabolism and inhibits tumor suppression while favoring tumor growth and proliferation respectively [26]. Moreover, PDAC can uptake extracellular proteins through micropinocytosis and lysosomal degradation to provide the necessary amino acids as an alternative nutrient source [28]. These dynamic changes in the regulation of specific proteins for the facilitation of essential amino acids that contribute to PDAC survival substantiate our results for the uneven distribution of amino acid contents in tumor and nontumor cells. Consequently, an analysis was performed to determine whether the amino acids undergoing selective exocytosis could be stressors to tumor cells when reintroduced. These selective exocytosis amino acids (positive Δ values, PSA) as stressors in tumor cells in a non-starvation manner, as illustrated schematically in FIG. 4.

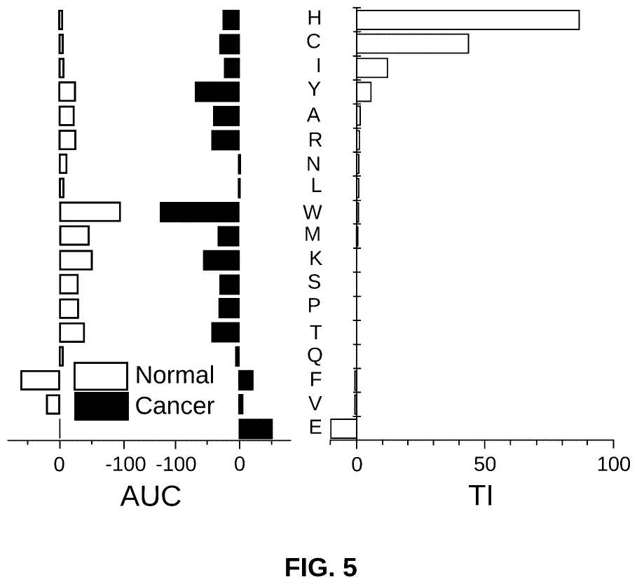

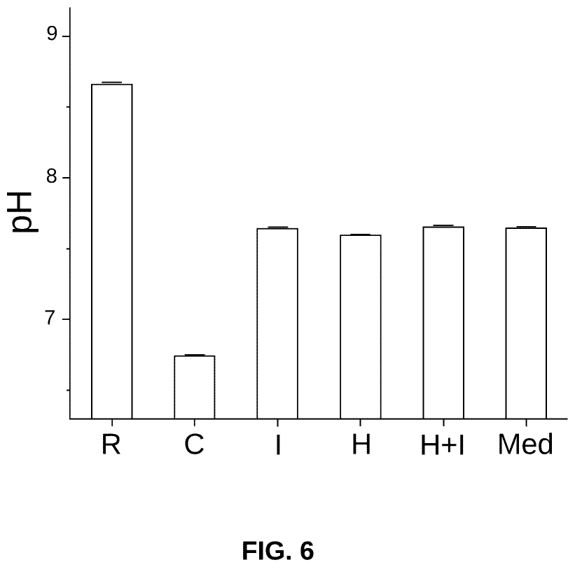

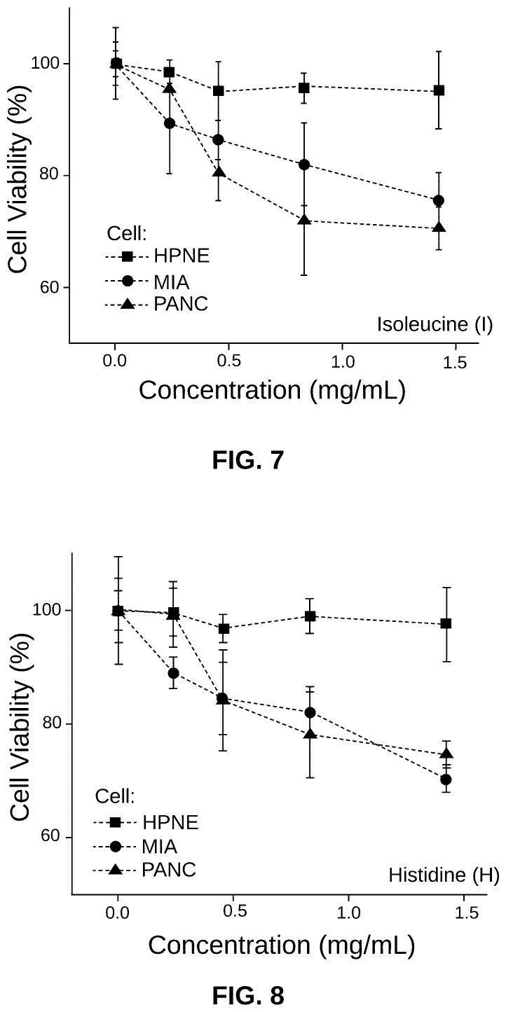

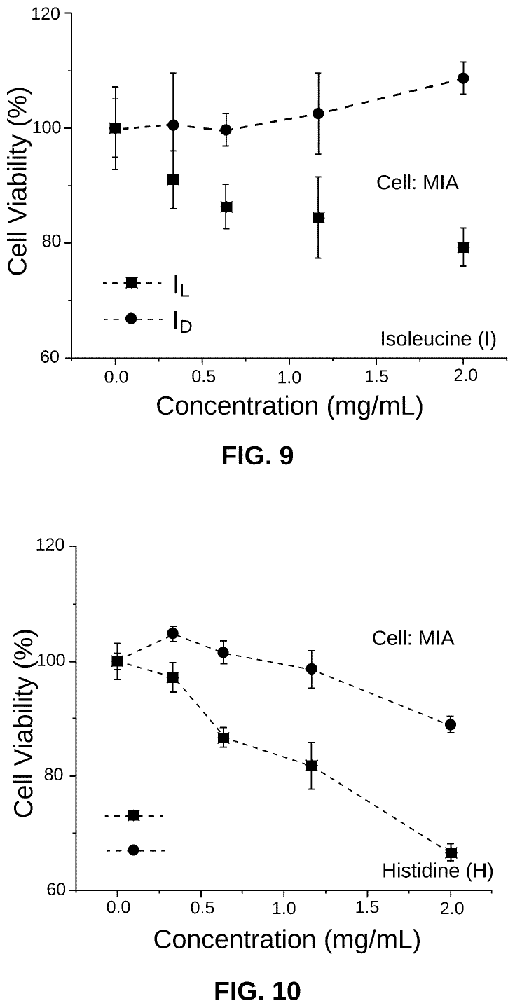

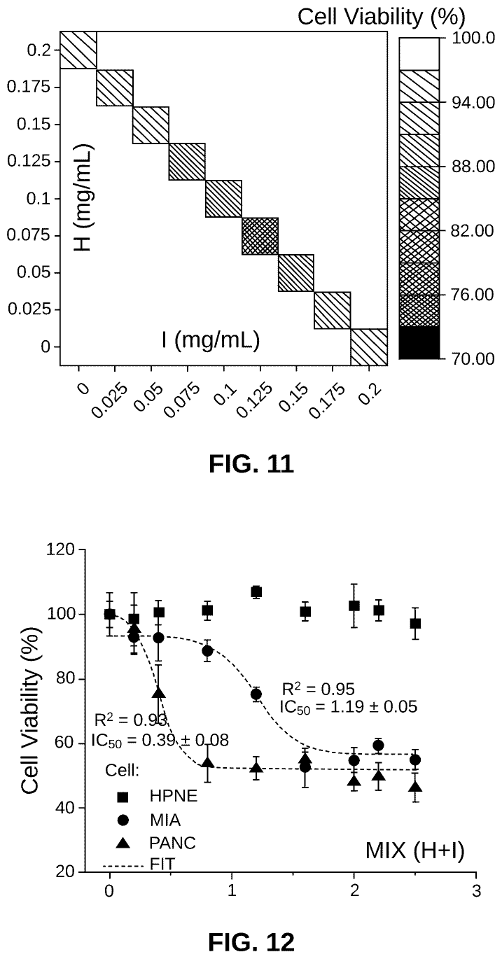

Validation of the effect of selective exocytosed amino acids as stressors to PDAC cells is illustrated in FIGS. 5-12. In particular, FIG. 5 shows area under the curve (AUC) and therapeutic index (TI) from amino acids cytotoxicity tests. FIG. 6 shows the pH change caused by amino acid addition with H and I exhibiting trivial impact to the media. FIG. 7 Illustrates the viability dose response of individual isoleucine (I). FIG. 8 illustrates the viability dose response of individual histidine (H) in the same cell lineage as Isoleucine (I) of FIG. 7. FIG. 9 shows a comparison of cell viability dose response between L and D form Isoleucine (I) amino acids. FIG. 10 shows a comparison of cell viability dose response between L and D form histidine (H). FIG. 11 illustrates synergetic efficacy of combining H and I treatment on tumor cells. Finally, FIG. 12 illustrates the cytotoxicity of H and I combined treatment, where “R” corresponds to arginine, “C” corresponds to cysteine, “H” corresponds to histidine, “I” corresponds to isoleucine, “Med” corresponds to medium only, “HPNE” again corresponds to hTert-HPNE (nonmalignant pancreas epithelial), “MIA” corresponds to MIA PaCa-2, and “PANC” corresponds to PANC-1 (malignant pancreas epithelial).

To address the question above, the individual amino acids exocytosed through tumor EVs (as shown in FIG. 3) were identified, and their cytotoxic effects were evaluated. The area under the curve (AUC) of each amino acid for cytotoxicity was measured, and the therapeutic index was calculated as the cytotoxicity (AUC) difference between nontumor and tumor cells (FIG. 5). The amino acids were then ranked by therapeutic index. It is recognized herein that, among the top-ranked amino acids, cysteine (C) and arginine (R) introduce significant pH change (FIG. 6), and cysteine (C) exhibited selective endocytosis (FIG. 3), making these proteins unsuitable for use in non-starvation treatment of cancers such as PDAC. Based on these results, histidine (H) and isoleucine (I) were chosen as the finalists for an amino acids approach to cancer treatment. In experimental studies, the tumor cells demonstrated a monotonic dose response to both amino acids. The effect of histidine and isoleucine treatment on nontumor cells showed no significant difference in cell viability, whereas treated tumor cells exhibited selective cytotoxic efficacy (FIGS. 7, 8). This result highlights the potential of amino acids treatment with selective efficacy in tumor cells, which may result in fewer adverse drug reactions (ADRs) or side effects in nontumor cells.

As stated above, the proteomics results appear to indicate an imbalance in the L-form amino acids excreted by the PDAC cells (FIGS. 2, 3). Thus, the functional efficacy of different isomeric forms of these amino acids were also tested. It should be noted that amino acids can occur in L- and D-forms, but only the L-forms are absorbable by cells. The study results demonstrated that the L-form of the amino acids had particularly significant effects on tumor cells, indicating their potential therapeutic value (FIGS. 9, 10).

Modern medicine often takes advantage of the combined use of several active agents to treat diseases, including cancer. Therefore, the synergetic efficacy of combining histidine and isoleucine for cancer treatment was also studied, and it was found that a remarkable efficacy burst may be obtained by combining these two amino acids, compared to using each amino acid individually in tumor cells treatment (FIG. 11). The optimized combination of the two amino acids (histidine and isoleucine), in a mass ratio of 2:1, has been validated for treatment to achieve maximal efficacy in vitro, and confirmed their highly selective cytotoxicity to the tumor cells (FIG. 12).

Histidine and isoleucine are essential amino acids that cannot be synthesized by mammalian cells but can be supplied through dietary intake and extracellular protein scavenging [29]. Genomic alterations by oncogenic transcription factors, such as c-MYC, HIF2α, and NOTCH, mediate high expression of LAT1 (SLC7A5) protein, which is involved in the intracellular influx and efflux movement of histidine and isoleucine that facilitate cancer development [30]. This fact suggests that the combined use of histidine and isoleucine (i.e., “AA” as referred to herein) may impact the expression of protein transporters and alter the metabolic milieu of tumor cells.

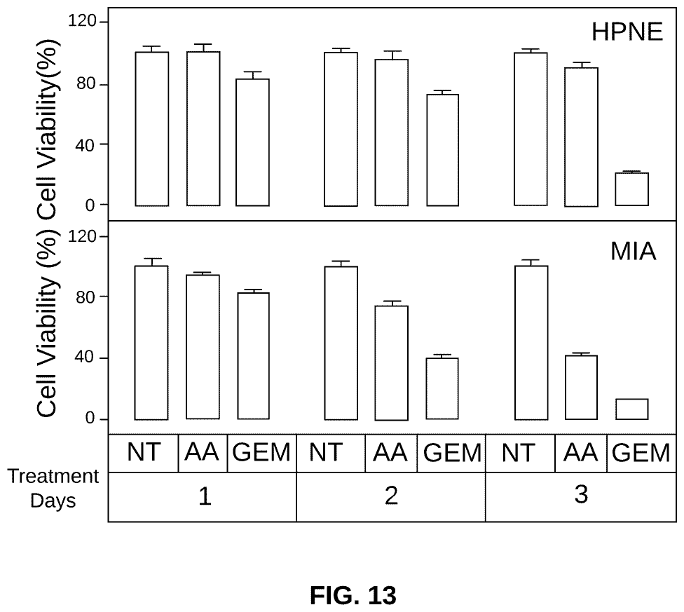

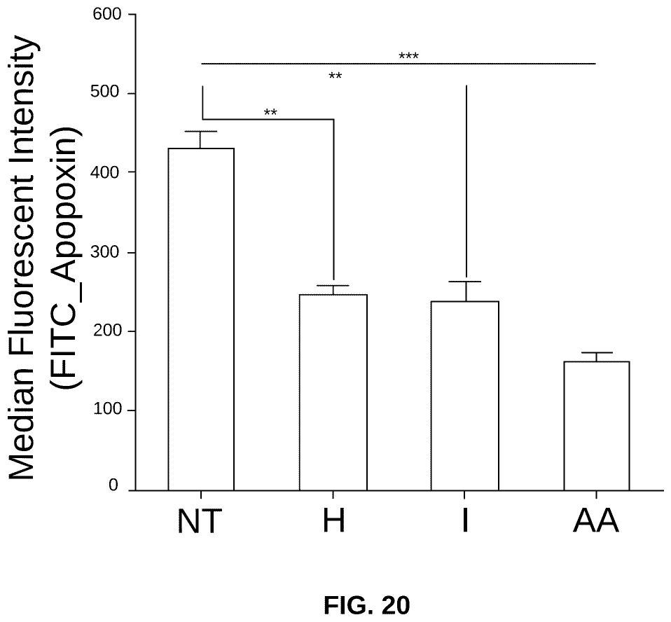

It is recognized herein that combined histidine and isoleucine (AA) treatment induces therapeutic effects and mediates cell death in PDAC. As described above, a non-starvation treatment using the combined amino acids (AA treatment) with selective exocytosis and delivering these amino acids as stressors to the tumor cells has been shown to be effective. A comparison of this AA treatment approach to a standard chemotherapy drug Gemcitabine (GEM) was also performed [31,32], as illustrated in FIGS. 13-26.

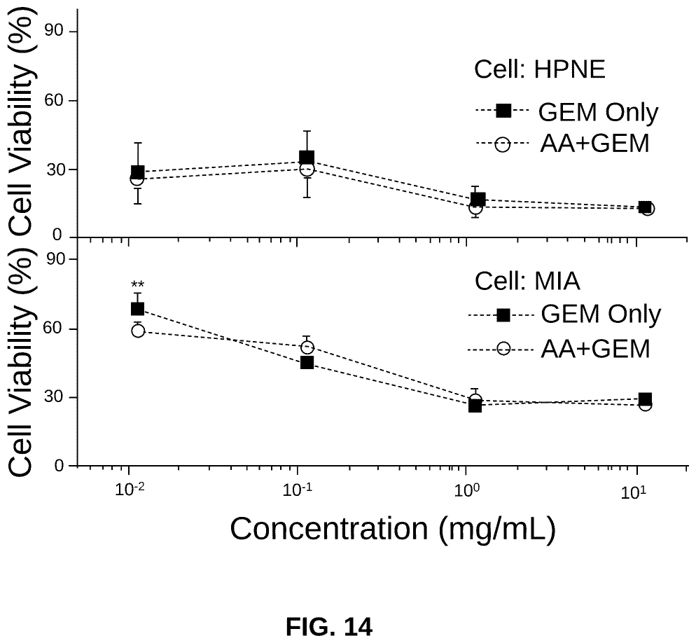

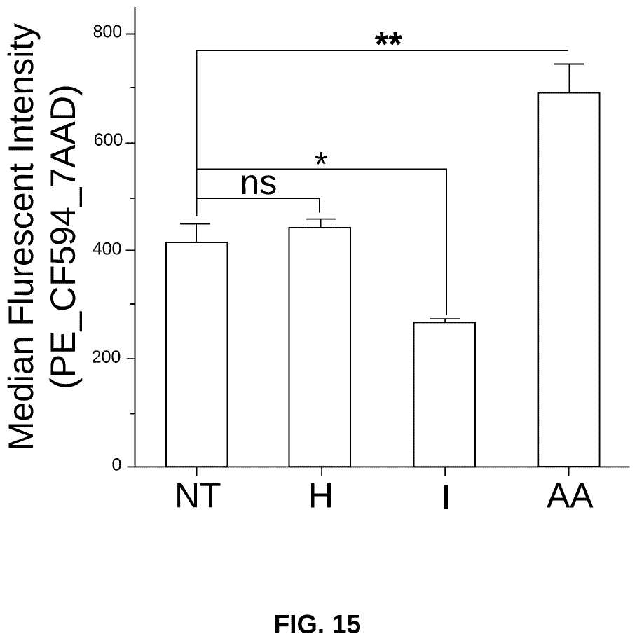

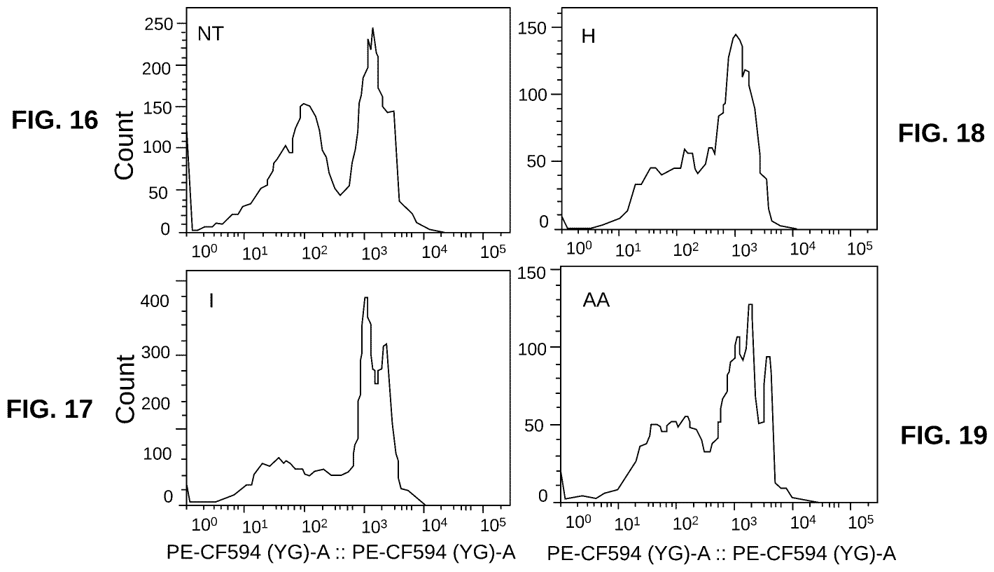

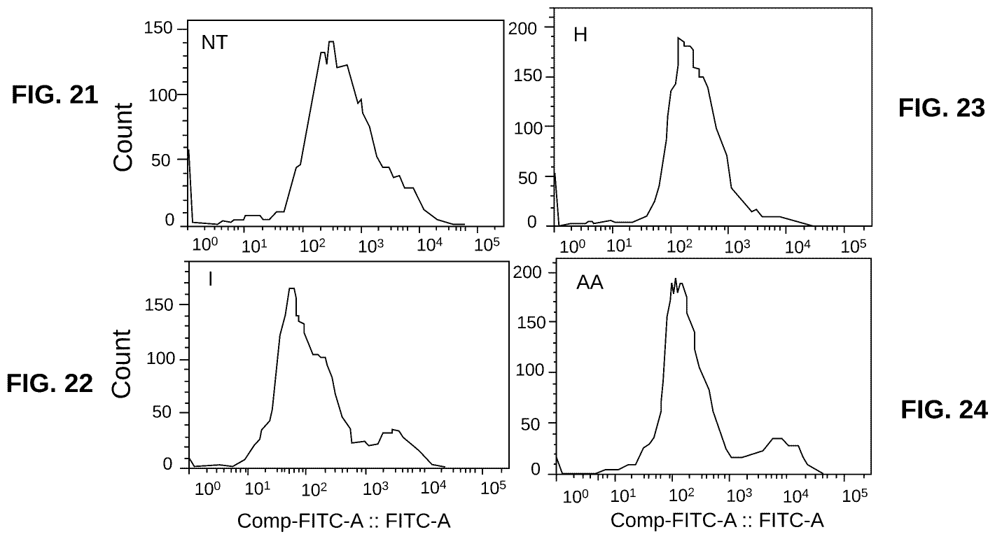

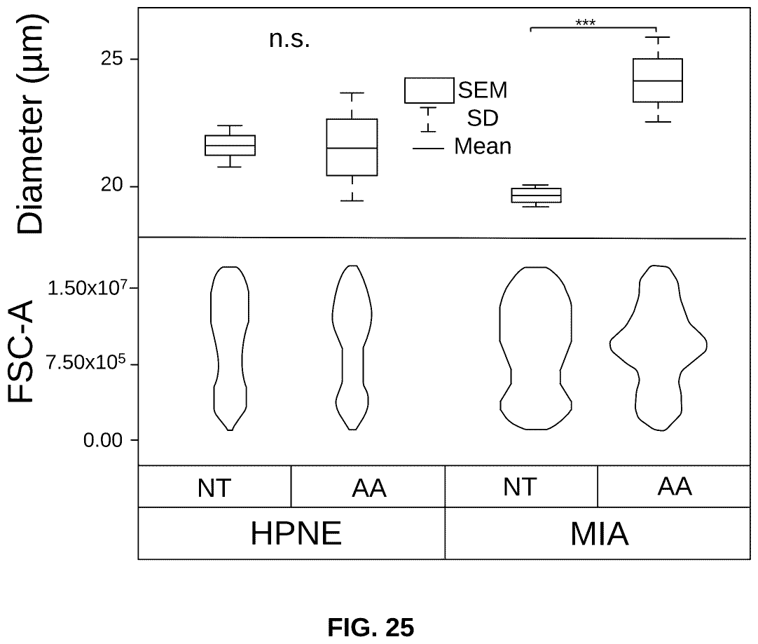

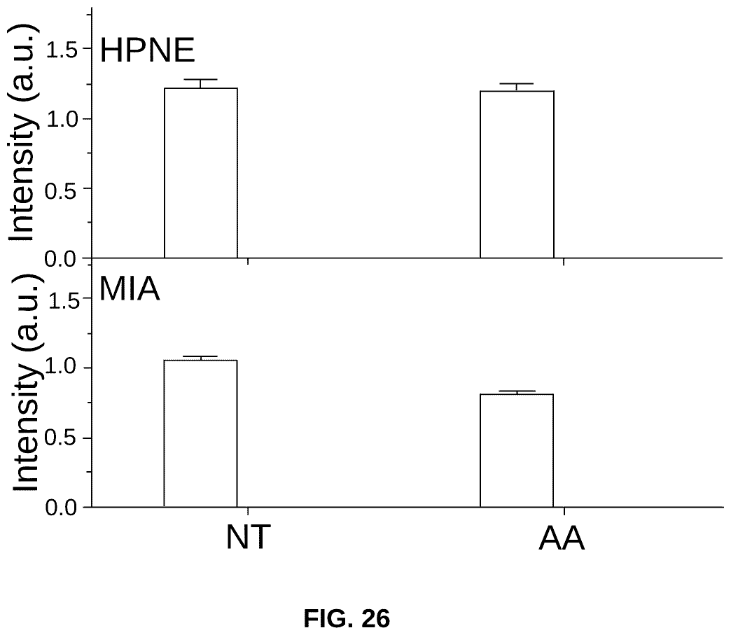

In particular, FIG. 13 shows a treatment efficacy comparison of “AA” (i.e., Histidine and Isoleucine) and “GEM” (i.e., gemcitabine). As shown in FIG. 13, adjuvant efficacy of combined AA+GEM treatment on tumor cells. FIG. 14 illustrates adjuvant efficacy of combined AA+GEM treatment on tumor cells. FIG. 15 shows a bar graph representing results of flowcytometric analyses with 7AAD/Apopxin/CytoCalcein-FITC assay for apoptosis and necrosis on tumor cells in a given set of treatment conditions, where “NT” represents no treatment control, “H” is treatment with histidine, “I” is treatment with isoleucine, and “AA” again is treatment with histidine and isoleucine. Error bars represent mean+SEM. FIGS. 16-19 show the measured counts corresponding to the AUC characterizations for NT, H, I, and AA, respectively, as summarized in FIG. 15. FIG. 20 shows a bar graph representing results of flowcytometric analyses with 7AAD/Apopxin/CytoCalcein-FITC assay for apoptosis and necrosis on tumor cells in a different set of treatment conditions. FIGS. 21-24 show the measured counts corresponding to the AUC characterizations for NT, H, I, and AA, respectively, as summarized in FIG. 20. FIG. 25 illustrates cell morphology changes upon AA treatment. FIG. 26 shows mitochondrial activity shifts due to AA treatment. HPNE: hTert-HPNE (nonmalignant pancreas epithelial); MIA: MIA PaCa-2 (malignant pancreas epithelial);

To reiterate, cellular-based assays were performed to compare cytotoxicity efficacy and explore possible mechanisms, we conducted cellular-based assays. FIG. 13 shows the time-dependent cytotoxicity of AA treatment compared with GEM. While GEM showed faster efficacy and is more tumoricidal effective, it exhibited a similar time-dependent cytotoxicity feature in nontumor cells, affirming the disadvantage of the standard chemotherapy regimen with elevated side effects due to destruction of healthy cells. In contrast, the AA treatment demonstrated selective time-dependent cytotoxicity to tumor cells without harm to normal cells, suggesting that this non-starvation approach may be safely used for effective and long-term treatment.

The synergistic effect of AA and GEM treatments is shown in FIG. 14. While no significant synergistic efficacy was observed in nontumor cells, low-dose GEM in combination with AA treatment showed enhanced efficacy in treating tumor cells, suggesting the potential of amino acids supplementation as an adjuvant regimen. This result also suggests that amino acid treatment may have a different tumoricidal mechanism from GEM.

It is known that histidine plays a crucial role in nucleotide synthesis, which is essential for tumor development. Alterations in the metabolic flux of histidine availability may affect the cellular pool of tetrahydrofolate, which influences methotrexate sensitivity to tumor cells [33]. Similarly, isoleucine plays a vital role in the modulation of the immune system against cancers by inducing β-defensin [34,35]. However, it is also known that GEM elicits its anticancer effect as a nucleotide analog interfering with DNA synthesis and ribonucleotide reductase [36]. The observed enhanced efficacy of the combined AA+GEM treatment may be attributed to its impact on nucleotide (DNA and/or RNA) metabolic regulation and immune system modulation.

A further study was performed to determine whether the treatment exerts its effects on tumor cells through apoptosis or necrosis. To this end, Apopoxin/7-AAD staining and flow cytometry analysis were performed on cancer cells subjected to AA treatment. The results showed that the AA treatment had the greatest therapeutic effect on tumor cells through necrosis, as demonstrated by the cell count and fluorescent intensity (FIGS. 15-24). As necrosis is characterized by morphological changes leading to plasma membrane rupture in an inflammatory-dependent manner [37], the effect of the combined AA treatment on tumor cell morphology, as compared to nontumor cells, were examined. Significant changes in tumor cell morphology, including increased cell diameter with inflammation, were observed, which further supports the occurrence of necrosis by the AA treatment (FIG. 25).

Drug-induced necrosis has been associated with mitochondrial dysfunction in many cancers [38]. It is also known that amino acids play a crucial role as nutrient sources and have a metabolic effect on PDAC. For example, an abnormal increase in mitochondrial activity via the Warburg effect is one of the characteristics responsible for PDAC tumor development and progression [39]. Thus, the effect of AA treatment on mitochondrial activity in PDAC tumor cells compared to nontumor cells was examined using fluorescent staining (FIG. 26). The results showed a significant decrease in mitochondrial activity in tumor cells due to the AA treatment, indicating an impact on metabolic activity that is critical to tumor cells. These findings suggest that the AA treatment mediates PDAC cell death through necrosis and its effect on metabolic activities is likely crucial to PDAC development. Identifying the metabolic pathway and therapeutic target of the combined AA treatment will provide further insight into its therapeutic effect.

Further experimentation has shown that XRN1 is a potential therapeutic target of the AA treatment. To explore the mechanism of action (MOA) of the AA treatment, a comparative proteomics analysis was performed before and after AA treatment application, followed by a pathway analysis using the Reactome Knowledgebase tool [40]. Four replicates each of treated and untreated cells were prepared, then the abundance ratio of untreated and treated cells proteins were calculated. The intensity ratio and p-value filters were set at Fold change >2 for upregulation and <0.2 for downregulation, and significant p-value <0.05 respectively, for significantly differentially expressed proteins. Enrichment scores were compared before and after AA treatment in PDAC cells.

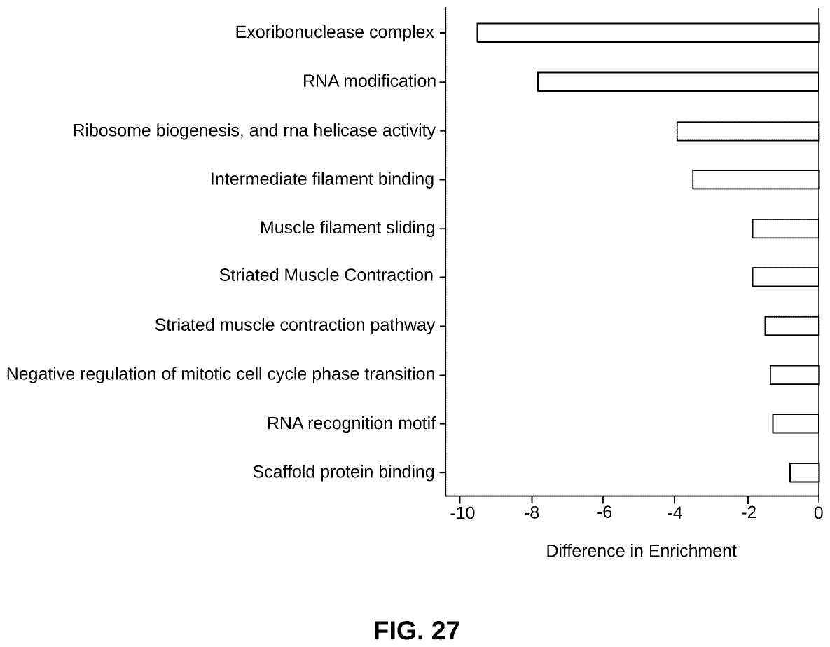

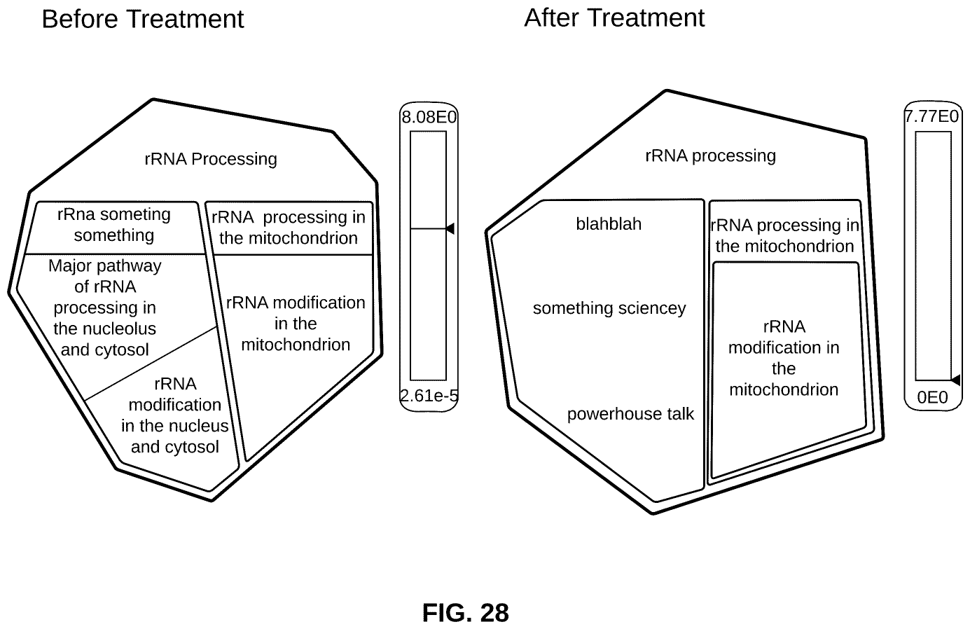

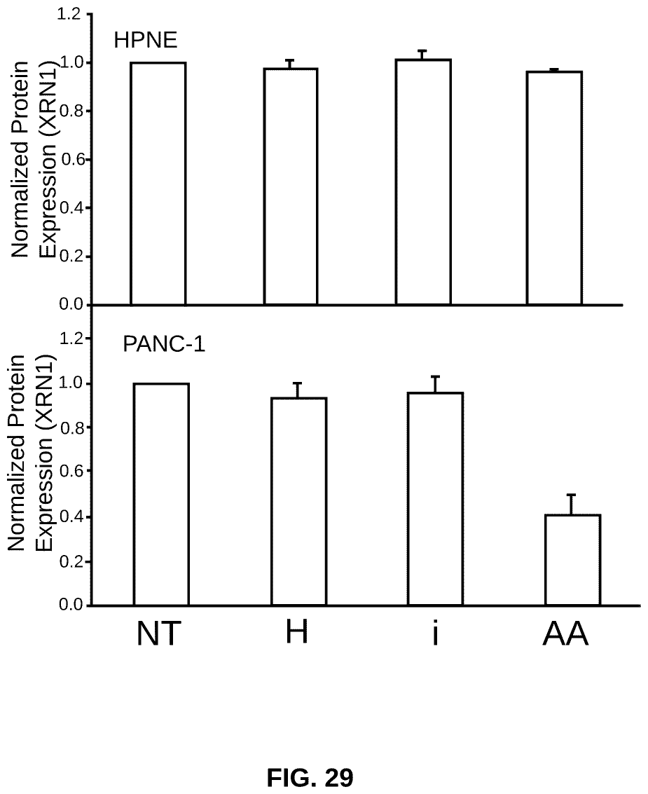

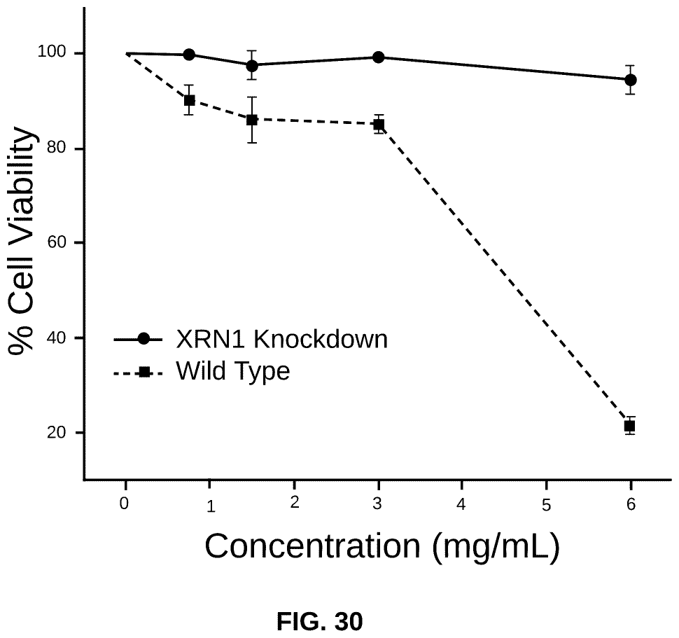

FIG. 27 shows differences in enrichment analysis before and after AA treatment. FIG. 28 shows a reactome analysis before and after AA treatment. FIG. 29 shows a bar graph comparison of Western blot expression analysis results of XRN1 protein in tumor and nontumor cells with combined AA treatment. FIG. 30 shows cytotoxicity effects of AA treatment on XRN1 knockdown cells and wild type. Again, “AA” stands for treatment with histidine and isoleucine, “HPNE” represents hTert-HPNE (nonmalignant pancreas epithelial), “XRN1” means XRN1 gene knockdown in PANC-1 tumor cells, and “PANC-1” means malignant pancreas epithelial and wildtype for the XRN1.

In particular, the pathways were ranked based on the enrichment difference before and after treatment, and the top-ranked amino acids are shown in FIG. 27. It was found that the exoribonuclease complex and RNA modification were the least enriched in the treatment group. In addition, a visual comparison using Reactome revealed that the RNA metabolic pathway was the most affected, and the XRN1 gene was ranked the highest of all. These results suggest that the treatment may affect the RNA metabolic pathway and perturb downstream post-transcriptional activity that is critical to protein expression in PDAC tumor cells. It is known that the XRN1 gene encodes the XRN1 exonuclease protein, which degrades mRNA. Notably, the analysis results may indicate that genetic alterations of the tumor cells and the exocytosis of histidine and isoleucine influence the degradation of mRNA transcripts, modulating the inflammatory response and providing a nutrient-rich milieu in the tumor cells. Therefore, it is speculated that reintroducing histidine and isoleucine through the AA treatment may repress the availability of the XRN1 protein, leading to the accumulation of mRNA transcripts that are toxic to the tumor cells.

Furthermore, the repression of the XRN1 gene by the AA treatment would lead to a reduction in the availability of the XRN1 protein. This theory was confirmed by western blot analysis, which showed reduced expression of the XRN1 protein in the AA treated group compared to other groups (FIG. 29). These results provide further evidence to the identification of XRN1 as a possible MOA for AA treatment.

To confirm the targeting of XRN1 by AA treatment for tumoricidal effect, XRN1 knockdown tumor cells were constructed (FIG. S3 in Appendix 2) and the cytotoxicity of AA treatment were compared to the wildtype cells. No significant changes in cell viability of the knockdown tumor cells were observed compared with the treated wildtype cells, which experienced a significant decrease in cell viability (FIG. 30). These results appear to further support the idea that XRN1 is the potential therapeutic target of the AA treatment and affects metabolic post-transcriptional activity in the tumor cells.

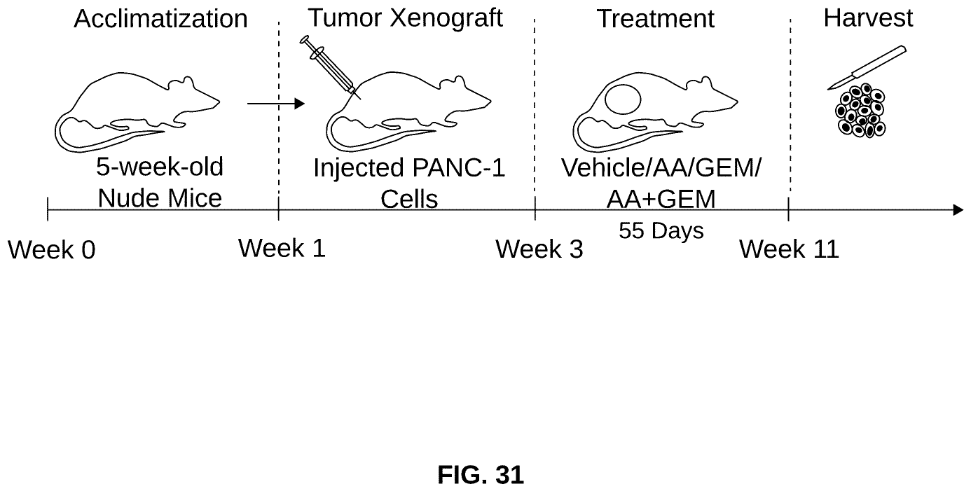

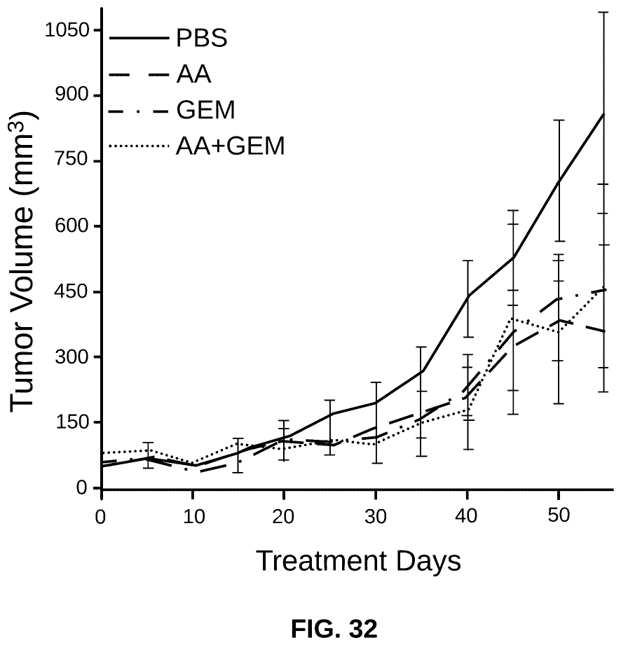

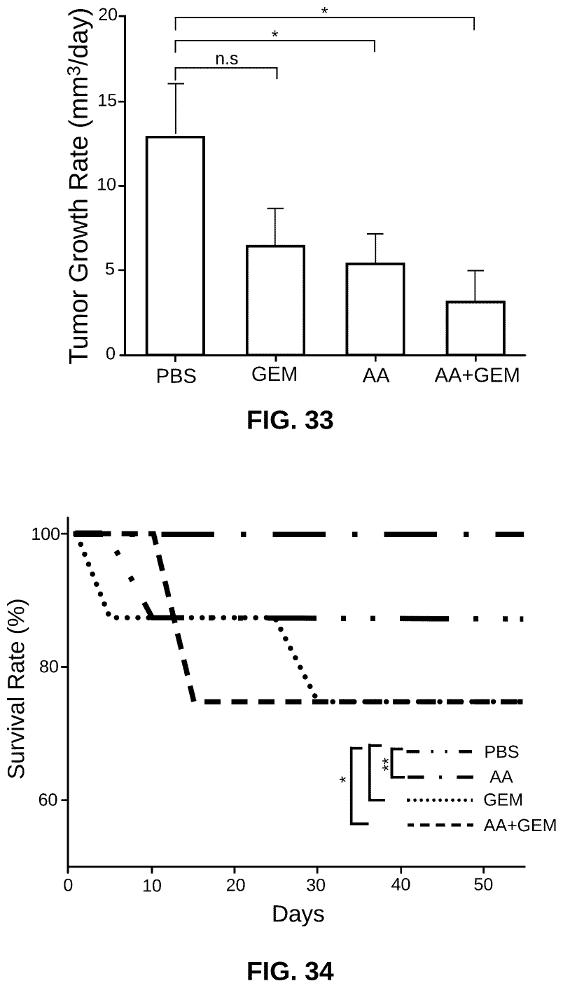

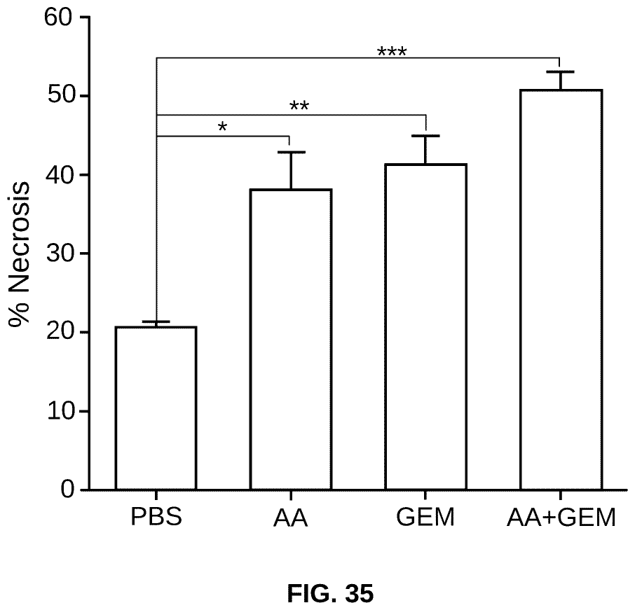

Further experimentation validated the hypothesis that AA treatment effectively inhibits PDAC tumor growth and extends mice survival, as illustrated below. In particular, FIG. 31 shows a simplified schematic of the in vivo experimental design (GEM: Gemcitabine; AA: histidine and isoleucine). FIG. 32 shows a graph comparison of tumor volume changes among the AA treatment and other treatment conditions. FIG. 33 shows a bar graph comparison of tumor growth rate among the AA treatment and other treatment conditions. FIG. 34 shows a comparison of survival rate among the AA treatment and other treatment conditions. FIG. 35 shows a comparison of percentage necrosis of the AA treatment and other treatment conditions evaluated using histology image analysis. Xenografts: n=5 for each treatment group. Tumor sections image sizes—1000 μm and 100 μm.

More particularly, to evaluate the therapeutic effect of AA treatment preclinically, an in vivo study using subcutaneous heterotopic mouse PDAC cancer modeling was performed (FIG. 31) and determined treatment efficacy using statistical assessment throughout the treatment. The treatment was administered orally. As shown in FIG. 32, all treatment groups (GEM, AA and AA+GEM) showed antitumor efficacy, consistent with the in vitro study (FIG. 13). Although AA+GEM displayed the highest tumor volume change from about 20 days through the course of 40 days of treatment, the antitumor efficacy of the three treatment groups (FIGS. 32, 33) appear to be comparable. Upon comparing the tumor growth rates affected by the treatment, AA+GEM demonstrated the highest antitumor growth effect compared to AA and GEM individually, which is in line with the in vitro study (FIG. 14) and suggests a synergistic efficacy of the AA and GEM treatment.

The survival rate of the subject mice were also analyzed and, interestingly, the AA treatment group showed a 100% survival rate (FIG. 34). While the GEM and AA+GEM treatment groups displayed varying survival rates at the beginning of the treatment, they eventually converged at 30 days. This finding further supports the selective toxicity of AA treatment on PDAC tumor cells in vivo. The high survival rate observed in the AA treatment group suggests that it may be a promising first-line therapy, while the synergistic antitumor effect of AA+GEM treatment highlights the potential of AA treatment as adjuvant therapy.

To further investigate the pathological efficacy of the treatment, hematoxylin/eosin (H&E) staining of tissue sections from the different treatment groups at the end of the in vivo study were performed. The resulting tumor tissue images were analyzed using the QuPath software [41]. Tissue sections from the untreated group exhibited a cellular solid nested pattern with hypercellularity, and nuclear pleomorphism, based on a Western blot assay. In contrast, the treated groups displayed varying degrees of necrosis as suggested by cytoplasmic vacuolation, reduced staining intensity, and infiltration of inflammatory cells with lower cellular density. Among the treatment groups, the AA+GEM treated group showed the most extensive ablation areas characterized by a reduction in tumor cellularity. Thus, the AA+GEM treatment group exhibited the highest level of necrosis (FIG. 35). These results are consistent with the in vitro study (e.g., as illustrated in FIGS. 15-26). Thus, it may be concluded that there is strong evidence of the therapeutic efficacy of the AA treatment for killing PDAC tumor cells, which may serve as alternative or combination therapy for combating pancreatic cancers.

The various studies described above have uncovered distinct differences in the proteome composition of PDAC tumor-derived EVs compared to nontumor cells [17]. Although mass spectrometry is a standard method for evaluating cellular proteins, most analyses end at only protein identification and quantification of unique peptides. In the aforedescribed studies, beyond the mass spectrometry analyses, the individual amino acids' distribution involved in the proteome of EVs and cells derived from PDAC were additionally characterized. The results indicate that tumor cells exhibit selective exocytosis of certain amino acids compared to nontumor cells. It has previously been known that amino acids play essential roles in providing nutrients, redox balance, energetic regulation, biosynthetic support, and homeostatic maintenance in various cells [42]. Moreover, amino acids have demonstrated metabolic reprogramming features to meet the nutritional demands of tumor cells to facilitate its progression dynamics [18]. Thus, tumor cells may exocytose specific amino acids to meet their metabolic requirements for growth. It is additionally recognized herein that highly selective, targeted treatment of cancer cells may be possible by using an amino acids-based approach. The studies performed above confirm that the exocytosed amino acids may act as stressors for PDAC tumor cells such that re-introduction of these specific amino acids may be used to inhibit the growth and progression of PDAC tumor cells.

Histidine and isoleucine are essential amino acids involved in nucleotide (DNA and RNA) metabolism and immune system modulation, respectively [43,44]. Some studies have utilized a starvation approach in amino acid treatment, limiting essential amino acids for disease progression [45-48]. However, the starvation approach is problematic as it alters mitochondria function, which may be detrimental to diseased patients[49] and subsequently contributes to the cachexia-anorexia syndrome experienced by most patients with advanced cancer [50]. Other studies have attempted the use of histidine supplementation in different cancers [43,51], and isoleucine supplementation in inhibiting tumor growth and angiogenesis in various cancers [52-54]. In contrast, the present disclosure provides a non-starvation amino acids treatment approach for pancreatic cancer by combining the use of histidine and isoleucine. This approach has several translational advantages, including selective toxicity against tumor cells, thus eliminating severe side effects exhibited by standard chemotherapeutic drugs, such as GEM. Additionally, amino acids are among the most accessible nutrients, with no side effects, making them particularly well-suited for counteracting malnutrition and cachexia in advanced-stage cancer patients, thus predisposing them to more effective treatment. Furthermore, amino acids may be applied as adjuvant therapy for various diseases, as discussed above, such as by providing amino acids treatment as an adjuvant therapy in combination with GEM, providing better efficacy in treating PDAC.

Moreover, the XRN1 protein has been identified as the potential therapeutic target in the above discussed amino acid-based treatment approach. As presented above, XRN1 protein is a highly conserved 5′-3′ exoribonuclease that regulates gene expression in eukaryotes by coupling nuclear DNA transcription to cytosolic mRNA decay [55]. XRN1 is involved in transcription, mRNA translation, and mRNA decay, and its modification plays a crucial role in post-transcriptional and translational activities that promote tumor development and chemoresistance [56,57]. Therefore, changes in the availability of the XRN1 protein and alterations in mRNA metabolism can lead to anomalous mRNA accumulation in PDAC tumor cells, which may trigger inflammatory responses and/or necrosis [55,57]. Thus, XRN1 protein is suitable as the potential therapeutic target of AA treatment, complementary to the targets for GEM, providing a novel approach for non-ADR treatment targeting mRNA translation and decay.

It is recognized herein that modifications to the described studies and embodiments above are possible. For example, in proteomics analysis, some genes may produce various forms of proteins (i.e., proteoforms) that may affect the overall amino acid estimation. While the necrosis-based tumoricidal effect was examined for the treatment in vitro, further studies may be necessary to confirm the detailed mechanism of action (MOA) and evaluate the pharmacodynamics/pharmacokinetics of the AA treatment in vivo. Additionally, while promising results of the treatment effect on tumor cell invasion and migration were observed in vitro (FIG. S4 in Appendix 2), the use of heterotopic subcutaneous mouse models for preclinical validation may not fully recapitulate the complexity of human tumorigenesis, particularly the interactions between tumor cells and the surrounding microenvironment. Therefore, further experimentation using engineered orthotopic mouse models that better mimic human pancreatic tumorigenesis may provide a more comprehensive evaluation of the treatment, including its impact on metastasis. Moreover, it is noted that, oral administration of the AA treatment was implemented in the studies described above due to the likelihood that intratumoral administration to the subcutaneous mouse model would behave similarly to an in vitro study and the potential risk of unexpected death associated with intravenous (IV) and intraperitoneal (IP) injections particularly for cancer patients. Oral intake also highlights the potential for diet modification as a preventive intervention. Further studies may be needed to validate oral administration and/or diet modification as a therapeutic method.

In summary, it is recognized herein that an amino acids-based treatment of cancer may be possible as a highly effective and targeted therapy to attack tumor cells. As an example, PDAC tumor cells have been observed to exocytose a unique group of amino acids via EVs, which act as stressors to the tumor cells. The above-described isoleucine and histidine diet-feeding represents a promising strategy for direct therapy and/or combination therapies with GEM for the deadly pancreatic duct carcinoma. Mechanistically, XRN1 has been characterized as the target of this amino acid-mediated RNA regulation pathways. Through different functional biochemistry, this AA treatment may have the potential for clinical application as direct or alternative therapy. Further supporting information may be found in reference [63] and its supporting supplementary files. All supporting information is considered a part of the present disclosure and incorporated herein by reference.

This treatment approach has several key advantages, such as targeting tumor metabolism specifically and reducing toxicity to normal cells. As an easy-to-administer non-starvation treatment, it is also less toxic and more sustainable in the long term, providing a promising targeted therapy for PDAC. While further research is needed to evaluate its clinical potential, our findings offer a promising new avenue in treating PDAC and a high-inspiration and transformative insight into cancer therapeutics. In particular, amino acid treatment is selective and effective against PDAC cells with virtually no side effects. This treatment approach may be used as an adjuvant with chemotherapy to enhance efficacy. Specific amino acids may also be combined with an optimal ratio to increase therapeutic effectiveness. Additionally, the treatment approach may be adapted for treatment of other types of cancer or subtype thereof by identifying the specific combination of amino acids and, optionally, adjuvant chemotherapy drug effective in discouraging aggregation of tumor growth for the particular cancer type or subtype. For example, EVs or equivalent for a specific cancer type may be identified and used to determine the amino acid imbalance that may be present in a sample taken from patients with the specific cancer type, and this information may be used in determining the amino acid treatment suitable for the specific cancer type.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms, even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding,” whether explicitly discussed or not, and, conversely, were there only disclosure of the act of “protruding,” such a disclosure should be understood to encompass disclosure of a “protrusion.” Such changes and alternative terms are to be understood to be explicitly included in the description.

### REFERENCES

1. Lockhart, C. et al. Treatment for pancreatic cancer: current therapy and continued progress. Gastroenterology, 128, 1642-1654 (2005).
2. McGuigan, A. et al. Pancreatic cancer: A review of clinical diagnosis, epidemiology, treatment and outcomes. World J Gastroenterol, 24, 4846-4861 (2018).
3. American Society of Clinical Oncology. Pancreatic Cancer: Statistics | Cancer.Net. (https://www.cancer.net/cancer-types/pancreatic-cancer/statistics) (2019).
4. Kamisawa, T. et al. Pancreatic Cancer. The Lancet, 388, 73-85 (2016).
5. Saung, M. T. et al. Current Standards of Chemotherapy for Pancreatic Cancer. Clin Ther, 39, 2125-2134 (2017).
6. Mallinson, C. N. et al. Chemotherapy in pancreatic cancer: Results of a controlled, prospective, randomised, multicentre trial. Br Med J, 281, 1589-1591 (1980).
7. Costa-Silva, B. et al. Pancreatic cancer exosomes initiate pre-metastatic niche formation in the liver. Nature Cell Biology, 2014 17:6 17, 816-826 (2015).
8. Yu, C. et al. Research of exosome in bone metastasis through dual aptamer recognition based entropy-driven amplification. Anal Biochem, 636, (2022).
9. Casari, I., et al. Extracellular vesicles derived from pancreatic cancer cells are enriched in the growth factor. Midkine Adv Biol Regul, 83, (2022).
10. Arance, E. et al. Determination of exosome mitochondrial DNA as a biomarker of renal cancer aggressiveness. Cancers (Basel), 14, (2022).
11. Wei, X. C., et al. Exosomes as potential diagnosis and treatment for liver cancer. World J Gastrointest Oncol, 14, 334-347 (2022).
12. Costa-Silva, B. et al. Pancreatic cancer exosomes initiate pre-metastatic niche formation in the liver. Nature Cell Biology, 2014 17:6 17, 816-826 (2015).
13. Yoshioka, Y. et al. Ultra-sensitive liquid biopsy of circulating extracellular vesicles using ExoScreen. Nat Commun, 5, 3591 (2014).
14. Andre, F. et al. MicroRNA signatures of tumor-derived exosomes as diagnostic biomarkers of ovarian cancer. Nat Biotechnol, 35, 883-91 (2012).
15. Chow, A. et al. Macrophage immunomodulation by breast cancer-derived exosomes requires Toll-like receptor 2-mediated activation of NF-κB. Sci Rep, 4, 5750 (2015).
16. Melo, S. A. et al. Glypican-1 identifies cancer exosomes and detects early pancreatic cancer. Nature, 523, 177-182 (2015).
17. Rasuleva, K. et al. β-Sheet Richness of the Circulating Tumor-Derived Extracellular Vesicles for Noninvasive Pancreatic Cancer Screening. ACS Sens, 6, 4489-4498 (2021).
18. Xu, R. et al. Reprogramming of Amino Acid Metabolism in Pancreatic Cancer: Recent Advances and Therapeutic Strategies. Front Oncol, 10, 572722 (2020).
19. Fauchon, M. et al. Sulfur sparing in the yeast proteome in response to sulfur demand. Mol Cell, 9, 713-723 (2002).
20. Pashler, A. L., et al. The roles of the exoribonucleases DIS3L2 and XRN1 in disease. Biochem Soc Trans, 44 (2016)
21. Ran, X.-B. et al. Targeting RNA Exonuclease XRN1 Potentiates Efficacy of Cancer Immunotherapy. Cancer Res, 83, 922-938 (2023).
22. Sun, D. et al. Dye-free spectrophotometric measurement of nucleic acid-to-protein ratio for cell-selective extracellular vesicle discrimination. Biosens Bioelectron, 179, 113058 (2021).
23. Colombo, M. et al. Analysis of ESCRT functions in exosome biogenesis, composition and secretion highlights the heterogeneity of extracellular vesicles. J Cell Sci, 126, 5553-5565 (2013).
24. Fauchon, M. et al. Sulfur Sparing in the Yeast Proteome in Response to Sulfur Demand. Molecular Cell, 9(4), 713-723 (2002).
25. Jones, S. et al. Core Signaling Pathways in Human Pancreatic Cancers Revealed by Global Genomic Analyses. Science, 321, 1801 (2008).
26. Qin, C. et al. Metabolism of pancreatic cancer: paving the way to better anticancer strategies. Molecular Cancer, 2020 19:1 19, 1-19 (2020).
27. Biankin, A., et al. Pancreatic cancer genomes reveal aberrations in axon guidance pathway genes. Nature, 491, 399-405 (2012).
28. Kamphorst, J. J. et al. Human Pancreatic Cancer Tumors Are Nutrient Poor and Tumor Cells Actively Scavenge Extracellular Protein. Cancer Res, 75, 544-553 (2015).
29. Neinast, M. D. et al. Quantitative Analysis of the Whole-Body Metabolic Fate of Branched-Chain Amino Acids. Cell Metab 29, 417-429.e4 (2019).
30. Wei, Z., et al. Metabolism of Amino Acids in Cancer. Front Cell Dev Biol, 8, 1628 (2021).
31. Conroy, T. et al. Current standards and new innovative approaches for treatment of pancreatic cancer. Eur J Cancer, 57, 10-22 (2016).
32. Manji, G. A., et al. Current and Emerging Therapies in Metastatic Pancreatic Cancer. Clin Cancer Res, 23, 1670-1678 (2017).
33. Kanarek, N. et al. Histidine catabolism is a major determinant of methotrexate sensitivity. Nature, 2018 559:7715 559, 632-636 (2018).
34. Gu, C., et al. Isoleucine Plays an Important Role for Maintaining Immune Function. Curr Protein Pept Sci, 20, 644-651 (2019).
35. Murata, K., et al. Isoleucine, an Essential Amino Acid, Prevents Liver Metastases of Colon Cancer by Antiangiogenesis. Cancer Res, 67, 3263-3268 (2007).
36. Ciccolini, J., et al. Pharmacokinetics and pharmacogenetics of Gemcitabine as a mainstay in adult and pediatric oncology: an EORTC-PAMM perspective. Cancer Chemother Pharmacol, 78, 1 (2016).
37. Alberts, B. et al. Programmed Cell Death (Apoptosis). Molecular Biology of the Cell, 4^{th} edition, (2002).
38. Bastian, A. et al. A Small Molecule with Anticancer and Antimetastatic Activities Induces Rapid Mitochondrial-Associated Necrosis in Breast Cancer. J Pharmacol Exp Ther, 353, 392-404 (2015).
39. Vyas, S., et al. Mitochondria and Cancer. Cell, 166, 555-566 (2016).
40. Gillespie, M. et al. The reactome pathway knowledgebase 2022. Nucleic Acids Res, 50, D687-D692 (2022).
41. Bankhead, P. et al. QuPath: Open source software for digital pathology image analysis. Sci Rep, 7, (2017).
42. Lieu, E. L., et al. Amino acids in cancer. Exp Mol Med, 15-30 (2020) doi:10.1038/s12276-020-0375-3.
43. Kanarek, N. et al. Histidine catabolism is a major determinant of methotrexate sensitivity. Nature, 2018 559:7715 559, 632-636 (2018).
44. Gu, C., et al. Isoleucine Plays an Important Role for Maintaining Immune Function. Curr Protein Pept Sci, 20, 644-651 (2019).
45. Kanarek, N. et al. Histidine catabolism is a major determinant of methotrexate sensitivity. Nature, 559, 632-636 (2018).
46. Fung, M. K. L., et al. Drug-induced amino acid deprivation as strategy for cancer therapy., J Hematol Oncol, 10, 1-18 (2017).
47. Lind, D. S. Arginine and Cancer. J Nutr, 134, 2837S-2841S (2004).
48. Choi, Y. K. et al. Targeting glutamine metabolism for cancer treatment. Biomol Ther (Seoul,) 26, 19-28 (2018).
49. Johnson, M. A. et al. Amino acid starvation has opposite effects on mitochondrial and cytosolic protein synthesis. PLoS One, 9, (2014).
50. Ragni, M., et al. Amino Acids in Cancer and Cachexia: An Integrated View. Cancers, 14, 5691 (2022).
51. Cheng, X. et al. Tumor-Specific Delivery of Histidine-Rich Glycoprotein Suppresses Tumor Growth and Metastasis by Anti-angiogenesis and Vessel Normalization. Curr Gene Ther, 14, 75-85 (2014).
52. Wang, H. et al. High dose isoleucine stabilizes nuclear PTEN to suppress the proliferation of lung cancer. Discover Oncology, 14, 1-17 (2023).
53. Murata, K. et al. Isoleucine, an Essential Amino Acid, Prevents Liver Metastases of Colon Cancer by Antiangiogenesis. Cancer Res, 67, 3263-3268 (2007).
54. Preciado, D., et al. Discovery of novel trihybrids based on salicylic acid/isoleucine/N-acylhydrazone: A promising therapeutic opportunity in colorectal cancer. J Appl Pharm Sci, 12, 10-020 (2022).
55. Blasco-Moreno, B. et al. The exonuclease Xrn1 activates transcription and translation of mRNAs encoding membrane proteins. Nature Communications, 2019 10:1 10, 1-15 (2019).
56. Akinlalu, A. O. et al. Recent developments in the significant effect of mRNA modification (M6A) in glioblastoma and esophageal cancer. Sci Afr, 17, (2022).
57. Ran, X.-B. et al. Targeting RNA Exonuclease XRN1 Potentiates Efficacy of Cancer Immunotherapy. Cancer Res, 83, (2023).
58. ImageJ MRI Wound Healing Tool. (https://github.com/MontpellierRessourcesImagerie/imagej_macros_and_scripts/wiki/Wound-Healing-Tool accessed 2025 Mar. 26).
59. Perez-Riverol, Y. et al. The PRIDE database resources in 2022: A hub for mass spectrometry-based proteomics evidences. Nucleic Acids Res, 50, D543-D552 (2022).
60. Rasuleva, K. et al. β-Sheet Richness of the Circulating Tumor-Derived Extracellular Vesicles for Noninvasive Pancreatic Cancer Screening. ACS Sens, 6, 4489-4498 (2021).
61. Kashif, M. et al. In vitro discovery of promising anti-cancer drug combinations using iterative maximisation of a therapeutic index. Sci Rep, 5, 1-13 (2015).
62. Gillespie, M. et al. The reactome pathway knowledgebase 2022. Nucleic Acids Res, 50, D687-D692 (2022).
63. Akinlalu, A. et al. Integrated proteomic profiling identifies amino acids selectively cytotoxic to pancreatic cancer cells. The Innovation, 5(3): 100626 (2024).

1. A method for selectively targeting tumor cells for a given strain of cancer, the method comprising:
providing a combination of a plurality of amino acids shown to discourage promotion of tumor growth for the given strain of cancer.

2. The method of claim 1, further comprising:
determining the plurality of amino acids by
identifying extracellular vesicles (EVs) involved in cell-cell signaling for tumor development for the given strain of cancer;
performing a proteomics analysis of the EVs in a patient sample to quantify relative abundance of individual amino acids present in the patient sample;
comparing the relative abundance of the individual amino acids from the patient sample to a known distribution of the individual amino acids present in a nonmalignant sample; and
if a significant imbalance in the relative abundance of the individual amino acids is present in the patient sample in comparison to the nonmalignant sample, then determining the individual amino acids exhibiting the significant imbalance as the plurality of amino acids suitable for discouraging promotion of tumor growth for the given strain of cancer.

3. The method of claim 1, further comprising supplementing the combination of the plurality of amino acids with a chemotherapy drug.

4. The method of claim 1, wherein the given strain of cancer is pancreatic ductile adenocarcinoma (PDAC).

5. The method of claim 4, wherein the combination of the plurality of amino acids targets elimination of exoribonuclease XRN1.

6. The method of claim 4, wherein the plurality of amino acids includes at least histidine and isoleucine.

7. The method of claim 6, further comprising supplementing the combination of the plurality of amino acids with gemcitabine.

8. The method of claim 1, wherein a therapeutic index (TI) of each one (i) of the plurality of amino acids is determined by
testing a plurality of cell lines for the given strain of cancer, and
calculating the TI using an equation:
$${TI}_{i} = \frac{{\sum }_{1}^{n} ( 1 - {v}_{i}^{Tumor} )}{n} - ( 1 - {v}_{i}^{N o n - t u m o r} ) where {v}_{i}^{Non - tumor}$$
is maximum cell viability value when treating nontumor tissue cells,
$${v}_{i}^{T u m o r}$$
is maximum cell viability value when treating tumor cells, and n is a total number of cell lines tested for the given strain of cancer.

9. A method for identifying presence of a given strain of cancer, the method comprising:
identifying extracellular vesicles (EVs) involved in cell-cell signaling for tumor development for the given strain of cancer;
performing a proteomics analysis of the EVs in a patient sample to quantify relative abundance of individual amino acids present in the patient sample;
comparing the relative abundance of the individual amino acids from the patient sample to a known distribution of the individual amino acids present in a nonmalignant sample; and
if a significant imbalance in the relative abundance of the individual amino acids is present in the patient sample in comparison to the nonmalignant sample, then determining the presence of the given strain of cancer.
